(19) United States
(12) Patent Application Publication
(10) Pub. No.: US 2025/0290018 A1
(43) Pub. Date: Sep. 18, 2025

(54) CARTRIDGES AND DEVICES FOR USE IN A SYSTEM FOR DELIVERY OF A PAYLOAD INTO A CELL

(71) Applicant: STEMCELL TECHNOLOGIES CANADA INC., Vancouver (CA)

(72) Inventor: Maisam DADGAR, Watertown, MA (US)

(21) Appl. No.: 18/868,410

(22) PCT Filed: May 25, 2023
(86) PCT No.: PCT/US2023/023567

§ 371 Date: Nov. 22, 2024

Related U.S. Application Data
(60) Provisional application No. 63/346,634, filed on May 27, 2022.

(51) Int. Cl. C12M 3/00; C12M 1/12; C12M 1/42

(52) U.S. Cl. CPC C12M 23/42; C12M 25/02; C12M 35/04

(57) ABSTRACT

A device for facilitating delivery of a payload to cells of a cell suspension includes a container and a cartridge configured to connect to an opening of the container when in an assembled position with respect to the container. The cartridge includes an input reservoir configured to receive a cell suspension. The cartridge includes a receiving portion configured to receive a constriction-containing element, wherein the constriction-containing element includes a constriction configured to perturb membranes of cells of the cell suspension to facilitate delivery of a payload to the cells, wherein the receiving portion is fluidly connected to the input reservoir to allow flow of the cell suspension from the input reservoir to the receiving portion, and wherein the receiving portion is fluidly connected to a cartridge outlet port to allow flow of the cell suspension from the receiving portion into an interior cavity of the container.

### CROSS-REFERENCE TO RELATED APPLICATIONS

This application claims priority to and benefit of U.S. Provisional Application No. 63/346,634, filed May 27, 2022, the entire contents of which is hereby incorporated by reference herein in its entirety, for all purposes.

### FIELD

The present disclosure relates to systems for delivery of a payloads into cells, and more specifically to cartridges and devices for holding constriction-containing elements having constricting channels or constricting pores, for use in systems for causing perturbations of cell membranes to allow passage of a payload through a cell membrane.

### BACKGROUND

The controlled delivery of various materials into cells is important in the developing medical field of cell therapy. For example, various research and therapeutic applications may include the delivery of peptides, nucleic acids, proteins, small molecules, and nanomaterials through cell membranes and into cells. As discussed in WO2013059343, WO2015023982, PCT/US2015/058489, PCT/US2015/060689, and PCT/US2016/13113, constricting microfluidic channels may be used to deliver compounds and other payloads into cells. As disclosed in PCT/US18/66295, tabletop laboratory and/or clinical systems may be configured to force a cell suspension through a cartridge, wherein the cartridge houses one or more constriction-containing elements (e.g., a part, piece, device, component, or the like, such as a microfluidic chip or a filter) having constricting channels or constricting pores, in order to cause perturbations in the membranes of the cells in the cell suspension.

### SUMMARY OF THE INVENTION

As explained above, some systems for intracellular payload delivery include cartridges are configured to house one or more constriction-containing elements (e.g., microfluidic chips or filters) having constricting channels or constricting pores, in order to cause perturbations in the membranes of the cells in a cell suspension when the cell suspension flows through the cartridge. However, these systems have cartridges that may be improved. For example, such systems do not allow for spatially-efficient and simple integration of cartridges with input reservoirs (e.g., reservoirs for holding fluid before it has passed through the cartridge) and output reservoirs (e.g., reservoirs for holding fluid after it has passed through the cartridge). Such systems rely, for example, on the use of flexible tubes for connecting cartridges to input reservoirs and/or output reservoirs, which may be provided for example in the form of flexible bags. These systems are not configured, for example, with respect to spatial efficiency, durability, simplicity, usability with existing laboratory equipment, and/or usability with robotics control systems.

Accordingly, there is a need for cartridges that have improved geometric configurations, improved spatial efficiency, improved durability, improved simplicity, improved usability with existing laboratory equipment, and/or improved usability with robotics control systems. The systems, methods, and techniques disclosed herein may address one or more of the above-identified needs.

Disclosed herein are devices for use with systems for delivery of payloads into cells. The devices include a cartridge configured to receive and hold a constriction-containing element such as a microfluidic chip or a microfluidic filter. Unlike cartridge systems that may be configured to be attached to an input reservoir by flexible tubing or by other fluidic components, the cartridges disclosed herein may include an input reservoir that is physically integrated with the cartridge itself. The input reservoir may be a reservoir configured to house cell suspension prior to its flow through the constriction-containing element. The cartridges disclosed herein may be provided in the form of a cap device configured to connect to and cover an opening of a container-such as a test tube, culture tube, sample tube, centrifuge tube, or the like. By using an integrated input reservoir, this eases integration with existing lab devices, such as readily available pressure sources. Additionally, the timing and potential volume reduction for using embodiments described herein may be particularly useful in a research use setting, where volumes are small, and consumables may be used quickly via numerous small volume experiments, for example. The container (e.g., tube) to which the cartridge may be attached as a cap may serve as an output reservoir for housing the cell suspension following processing through the constriction-containing element. In some embodiments, when the cartridge is in the attached position with the container (e.g., tube), a portion of the cartridge may be disposed inside an interior cavity of the container, for example by protruding downward into the interior of a test tube. This configuration eases the retrieval of a tube container, and may provide additional ease of use in a research setting.

Thus, when the cartridge is attached to an open end of a container such as a laboratory tube, the device formed by the combination of the cartridge and the container may include an input reservoir, a receiving portion configured to receive and hold the constriction-containing element, and an output reservoir provided in the form of the container such as a laboratory tube. All three components may be in fluid communication with one another to allow flow of the cell suspension from the input reservoir to the receiving portion (and into and out of a constriction-containing element housed therein) and then from the receiving portion to the container (e.g., tube). Following flow of the cell suspension into the container (e.g., tube), the cartridge may be detached from the opening of the container and the container (housing the processed cell suspension) may be used for further processing of the cell suspension, may be capped, and/or may be stored in any suitable manner. When the container is provided in the form of a standard laboratory tube, the tube may be easily used for downstream processing by one or more additional standard-compatible devices, caps, and/or storage devices.

Some embodiments are directed to a device for facilitating delivery of a payload to cells of a cell suspension, the device including a container including an interior cavity and an opening, and a cartridge configured to connect to the opening of the container when in an assembled position with respect to the container. The cartridge includes an input reservoir configured to receive a cell suspension, and a receiving portion configured to receive a constriction-containing element. The constriction-containing element includes a constriction configured to perturb membranes of cells of the cell suspension to facilitate delivery of a payload to the cells. The receiving portion is fluidly connected to the input reservoir to allow flow of the cell suspension from the input reservoir to the receiving portion, and the receiving portion is directly fluidly connected to a cartridge outlet port to allow flow of the cell suspension from the receiving portion into the interior cavity of the container.

In some embodiments, when the cartridge is in the assembled position with respect to the container, a portion of the receiving portion of the cartridge is disposed inside the interior cavity of the container. In some embodiments, when the cartridge is in the assembled position with respect to the container, a portion of the input reservoir of the cartridge is disposed inside the interior cavity of the container.

In some embodiments, the device includes a cover configured to releasably attach to the receiving portion of the cartridge to hold the constriction-containing element in place when the constriction-containing element is received by the receiving portion. In some embodiments, the cover is configured to slidably attach and detach to the receiving portion of the cartridge. In some embodiments, the cover is configured to slide in a direction perpendicular to a direction in which layers of the constriction-containing element are stacked with one another. In some embodiments, the cover is configured such that, when the cartridge is in the assembled position with respect to the container, the cover is prevented by an interior wall of the container from detaching from the receiving portion.

In some embodiments, the cartridge includes an input reservoir cover that is movable between an open position and a closed position to selectively expose and enclose an interior of the input reservoir.

In some embodiments, the cartridge includes a first o-ring disposed against a surface of the input reservoir cover that faces away from the interior of the input reservoir when the input reservoir cover is in the closed position. In some embodiments, the cartridge includes a second o-ring disposed between the input reservoir cover and a body portion of the cartridge.

In some embodiments, the input reservoir cover includes a filter configured to prevent backflow of the cell suspension. In some embodiments, the cartridge has an overall fluid throughput of greater than 0.05 L/min. In some embodiments, the cartridge is configured to allow flow of the cell suspension through the cartridge at a pressure of greater than 100 PSI.

Some embodiments are directed to a device for facilitating delivery of a payload to cells of a cell suspension. The device includes a container including an interior cavity and an opening, and a cartridge configured to connect to the opening of the container when in an assembled position with respect to the container, the cartridge including an input reservoir configured to receive a cell suspension, and a receiving portion. The device includes a constriction-containing element. The constriction-containing element includes a constriction configured to perturb membranes of cells of the cell suspension to facilitate delivery of a payload to the cells. The receiving portion of the cartridge is fluidly connected to the input reservoir and to the constriction-containing element, such that the cell suspension is allowed to flow from the input reservoir through the receiving portion and into the constriction-containing element, and such that the cell suspension is allowed to flow out of the constriction-containing element through the receiving portion and into the interior cavity of the container.

In some embodiments, the device includes a gasket disposed between the receiving portion and the constriction-containing element. In some embodiments, the constriction-containing element includes a microfluidic chip including a plurality of constrictions that perturb the membranes of the cells. In some embodiments, the constriction-containing element has a footprint of less than 5 mm by 5 mm.

In some embodiments, when the cartridge is in the assembled position with respect to the container, a portion of the receiving portion of the cartridge is disposed inside the interior cavity of the container. In some embodiments, when the cartridge is in the assembled position with respect to the container, a portion of the input reservoir of the cartridge is disposed inside the interior cavity of the container.

In some embodiments, the cartridge further includes a cover configured to releasably attach to the receiving portion of the cartridge to hold the constriction-containing element in place when the constriction-containing element is received by the receiving portion. In some embodiments, the cover is configured to slidably attach and detach to the receiving portion of the cartridge. In some embodiments, the cover is configured to slide in a direction perpendicular to a direction in which layers of the constriction-containing element are stacked with one another. In some embodiments, the cover is configured such that, when the cartridge is in the assembled position with respect to the container, the cover is prevented by an interior wall of the container from detaching from the receiving portion.

In some embodiments, the cartridge includes an input reservoir cover that is movable between an open position and a closed position to selectively expose and enclose an interior of the input reservoir.

In some embodiments, the cartridge includes a first o-ring disposed against a surface of the input reservoir cover that faces away from the interior of the input reservoir when the input reservoir cover is in the closed position. In some embodiments, the cartridge includes a second o-ring disposed between the input reservoir cover and a body portion of the cartridge.

In some embodiments, the input reservoir cover includes a filter configured to prevent backflow of the cell suspension. In some embodiments, the cartridge has an overall fluid throughput of greater than 0.05 L/min. In some embodiments, the cartridge is configured to allow flow of the cell suspension through the cartridge at a pressure of greater than 100 PSI.

Some embodiments are directed to a cartridge for facilitating delivery of a payload to cells of a cell suspension, the cartridge including an input reservoir configured to receive a cell suspension, a connection portion configured to connect to an opening to an interior cavity of a container, and a receiving portion configured to receive a constriction-containing element. The constriction-containing element includes a constriction configured to perturb membranes of cells of the cell suspension to facilitate delivery of a payload to the cells. The receiving portion is fluidly connected to the input reservoir to allow flow of the cell suspension from the input reservoir to the receiving portion, and the receiving portion is fluidly connected to a cartridge outlet port to allow flow of the cell suspension out of the cartridge.

In some embodiments, when the cartridge is connected in an assembled position with respect to the container, a portion of the receiving portion of the cartridge is disposed inside the interior cavity of the container. In some embodiments, when the cartridge is connected in an assembled position with respect to the container, a portion of the input reservoir of the cartridge is disposed inside the interior cavity of the container.

In some embodiments, the cartridge further includes a cover configured to releasably attach to the receiving portion of the cartridge to hold the constriction-containing element in place when the constriction-containing element is received by the receiving portion. In some embodiments, the cover is configured to slidably attach and detach to the receiving portion of the cartridge. In some embodiments, the cover is configured to slide in a direction perpendicular to a direction in which layers of the constriction-containing element are stacked with one another. In some embodiments, the cover is configured such that, when the cartridge is connected in an assembled position with respect to the container, the cover is prevented by an interior wall of the container from detaching from the receiving portion.

In some embodiments, the cartridge includes an input reservoir cover that is movable between an open position and a closed position to selectively expose and enclose an interior of the input reservoir. In some embodiments, the cartridge includes a first o-ring disposed against a surface of the input reservoir cover that faces away from the interior of the input reservoir when the input reservoir cover is in the closed position. In some embodiments, the cartridge includes a second o-ring disposed between the input reservoir cover and a body portion of the cartridge.

In some embodiments, the input reservoir cover includes a filter configured to prevent backflow of the cell suspension. In some embodiments, the cartridge has an overall fluid throughput of greater than 0.05 L/min. In some embodiments, the cartridge is configured to allow flow of the cell suspension through the cartridge at a pressure of greater than 100 PSI. In some embodiments, the cartridge has an overall fluid throughput of less than 0.5 L/min. In some embodiments, the cartridge is configured to allow flow of the cell suspension through the cartridge at a pressure of less than 200 PSI.

In some embodiments, any one or more of the features, characteristics, or elements discussed above with respect to any of the embodiments may be incorporated into any of the other embodiments mentioned above or described elsewhere herein. In some embodiments, any one or more of the features, characteristics, or elements discussed elsewhere in this disclosure may be incorporated into any one or more of the embodiments mentioned above.

### BRIEF DESCRIPTION OF THE DRAWINGS

FIG. 1 illustrates a system for delivering a payload to a cell, in accordance with some embodiments.

FIGS. 2A-2E illustrate various views of a device including a cartridge and a container, for use in a system for delivering a payload to a cell, in accordance with some embodiments.

FIGS. 3A-3B illustrate views of a cartridge for use in a system for delivering a payload to a cell, in accordance with some embodiments.

FIGS. 4A-4B illustrate views of a chip and gasket assembly for use with a cartridge for use in a system for delivering a payload to a call, in accordance with some embodiments.

### DETAILED DESCRIPTION OF THE INVENTION

Described below are exemplary embodiments of cartridges for use in systems for partially or fully automated intracellular payload delivery, as well as associated devices, systems, methods, and techniques.

The following description sets forth exemplary systems, methods, techniques, parameters, and the like. It should be recognized, however, that such description is not intended as a limitation on the scope of the present disclosure but is instead provided as a description of exemplary embodiments.

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The term “pore” as used herein refers to an opening, including without limitation, a hole, tear, cavity, aperture, break, gap, or perforation within a material. In some examples, (where indicated and/or where it would be clear, in light of the disclosure, to a person of skill in the art) the term refers to a pore within a surface of the present disclosure. In other examples, (where indicated and/or where it would be clear, in light of the disclosure, to a person of skill in the art) a pore can refer to a pore in a cell membrane.

The term “filter” as used herein refers to a porous article that allows selective passage through the pores. In some examples the term refers to a surface or membrane containing pores.

Although the description herein uses terms first, second, etc. to describe various elements, these elements should not be limited by the terms. These terms are only used to distinguish one element from another.

For any of the structural and functional characteristics described herein, methods of determining these characteristics are known in the art.

All references cited herein, including patent applications and publications, are incorporated by reference in their entirety.

### Cartridges for Use in Intracellular Payload Delivery Systems

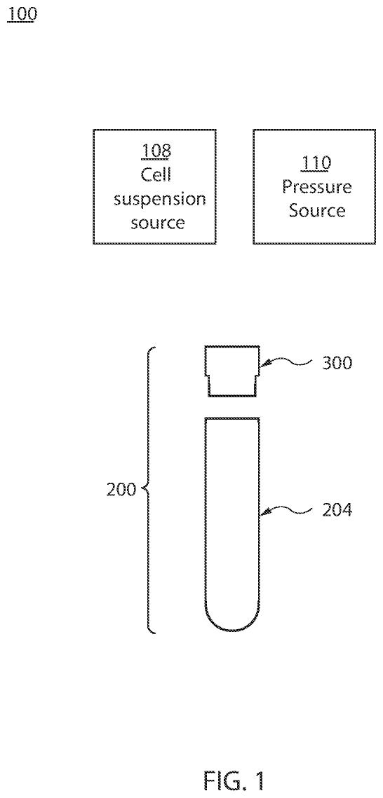

FIG. 1 shows a schematic diagram of intracellular payload delivery system 100.

In some embodiments, system 100 may share any one or more characteristics with any one of the systems described in WO 2019/126212 and/or in WO 2020/210162, the entire contents of each of which are hereby incorporated by reference. In short, an intracellular payload delivery system may enable the delivery of a payload into cells by forcing the cells to flow through a constriction such as a narrow microfluidic channel or a narrow pore, thereby perturbing the membranes of the cells and allowing the payload to enter the cells.

While WO 2020/210162 describes a system for intracellular payload delivery, the system described in WO 2020/210162 relies on a cartridge that is fluidically connected by flexible tubing and configured to receive flow of a cell suspension from a preparation vessel; and that is fluidically connected by flexible tubing and configured to direct flow of the cell suspension to an output bag. As described herein, the cartridges disclosed herein may differ from the cartridges described in WO 2020/210162 in that the cartridge described herein may be provided in the form of a cap device configured to connect to a container such as a test tube, wherein the cartridge may include an input reservoir that is formed as an integral part of the cap device.

In some embodiments of system 100, constrictions such as narrow microfluidic channels or narrow pores may be provided in microfluidic chips or filters (which may be referred to as constriction-containing elements), which may be attached in fluid communication to system 100. In some embodiments, the microfluidic chips or filters (or any other element comprising one or more constrictions configured to perturb the membranes of the cells) may be provided and fluidly connected to a system such as system 100 by way of a cartridge. A cartridge may be any device configured to house an element comprising a constriction, such as a microfluidic chip or a filter, and/or to facilitate the fluid connection of the element (e.g., chip or filter) to another portion of an intracellular payload delivery system, such as system 100.

As shown in FIG. 1, system 100 may comprise cartridge 300, container 204, cell suspension source 108, and pressure source 110. Cartridge 300 and container 204 may together form device 200.

Cartridge 300 may be formed as a cap for container 204, and may be configured to attach to container 204 by way of a threaded connection, a press connection, a snap-fit connection, or any other suitable attachment means. When in the attached position with respect to container 204, cartridge 300 may cover an opening of container 204, which in the arrangement shown in FIG. 1 may be an opening in the top of container 204.

Container 204 may be any suitable container having one or more outer walls enclosing an interior cavity, and having at least one opening to the interior cavity, wherein the opening is configured to mate with cartridge 300. In some embodiments, container 204 may be a tube such as a test tube, culture tube, sample tube, centrifuge tube, or the like. In some embodiments, container 204 may be an Eppendorf tube.

Cartridge 300 may include both an input reservoir and a receiving portion. The input reservoir may comprise a cavity formed within the cartridge and configured to house a cell suspension before it is caused to flow through a constriction-containing element such as a microfluidic chip or filter. The receiving portion may comprise a portion of cartridge 300 that is configured to receive and house the constriction-containing element itself. The input reservoir and the receiving portion may be fluidly connected to one another, for example by one or more internal channels, such that the cell suspension may flow from the input reservoir to the receiving portion and into the constriction-containing element housed in the receiving portion. After flowing through the constriction-containing element, the cell suspension may flow out of the constriction containing element and back into the receiving portion. The cell suspension may then flow, for example by one or more internal channels, from the receiving portion out of the cartridge and into the cavity of the container.

System 100 may further comprise cell suspension source 108, which may comprise any source (e.g., reservoir, tank, vessel, etc.) from which cartridge 300 may receive the cell suspension. In some embodiments, an input opening to the input reservoir of cartridge 300 may be configured to be fluidly connected (e.g., by being selectively connectable and disconnectable) to cell suspension source 108 such that the cell suspension may flow from cell suspension source 108 to the input reservoir of cartridge 300.

System 100 may further comprise pressure source 110, which may comprise any source configured to apply pressure to the cell suspension inside input reservoir of cartridge 300. In some embodiments, pressure source 110 may be provided as part of the same fluid source mechanism as cell suspension source 108. In some embodiments, pressure source 110 may be configured to be connected (e.g., by being selectively connectable and disconnectable) the input reservoir of cartridge 300 in order to apply pressurized gas, pressurized liquid, or/or mechanical pressure to the cell suspension within the input reservoir of cartridge 300. The pressure applied to the cell suspension within the input reservoir of cartridge 300 may cause the cell suspension to flout out of the input reservoir and through the one or more constrictions of the constriction-containing element housed in the receiving portion of cartridge 300. In some embodiments, pressure source 110 may share any one or more characteristics in common with the pressure control modules and/or other pressure control components described in WO 2019/126212. As above, WO 2019/126212 is incorporated by reference herein in its entirety, for all purposes.

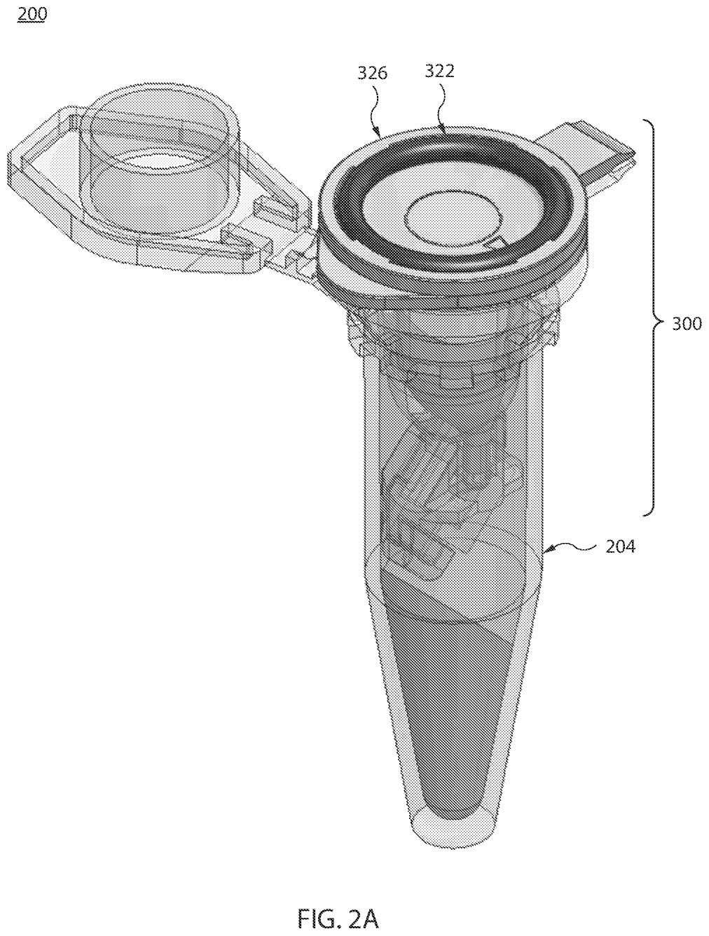

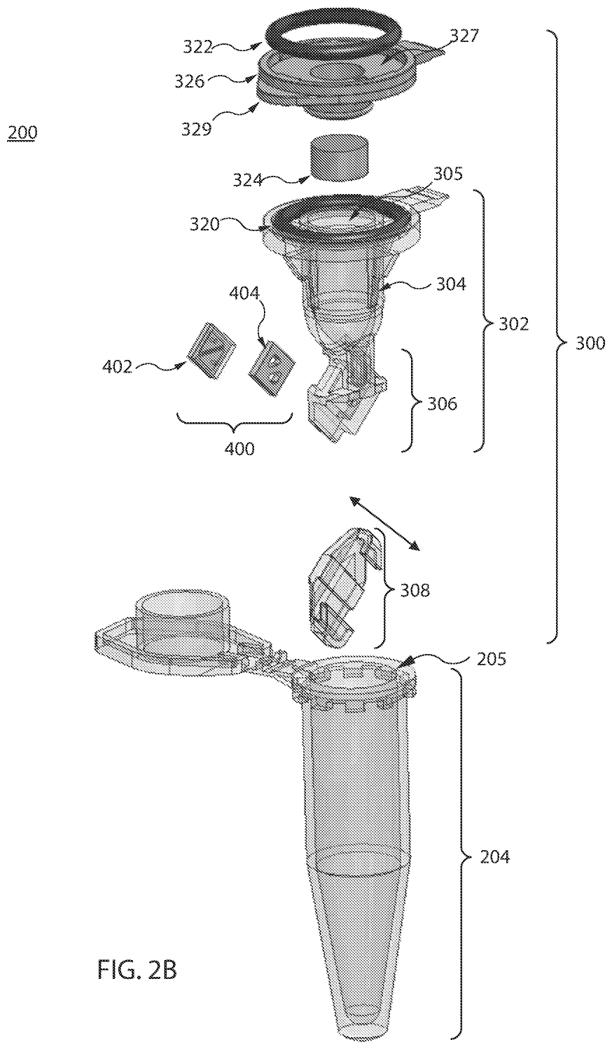

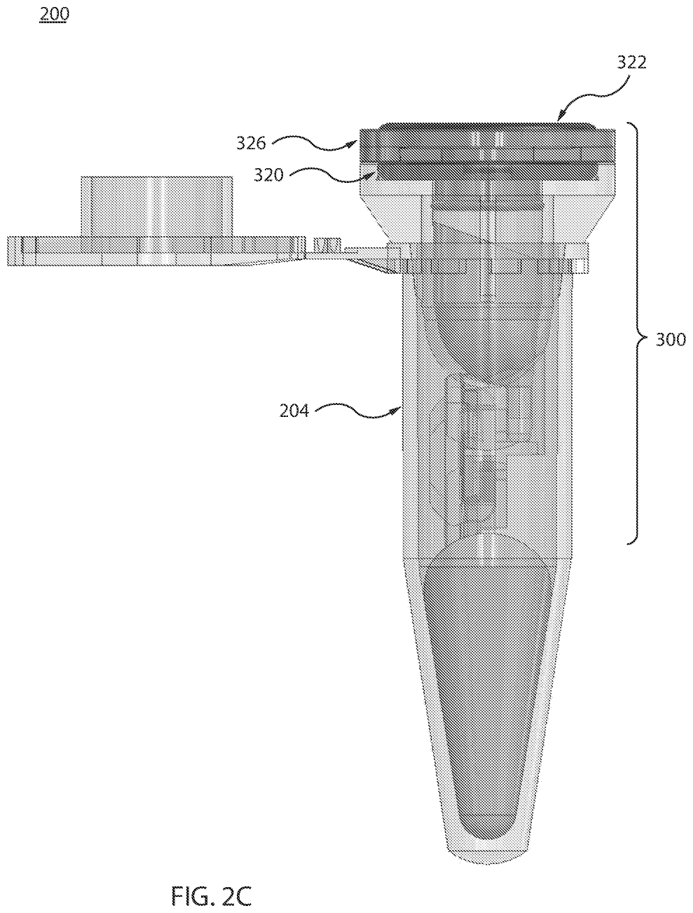

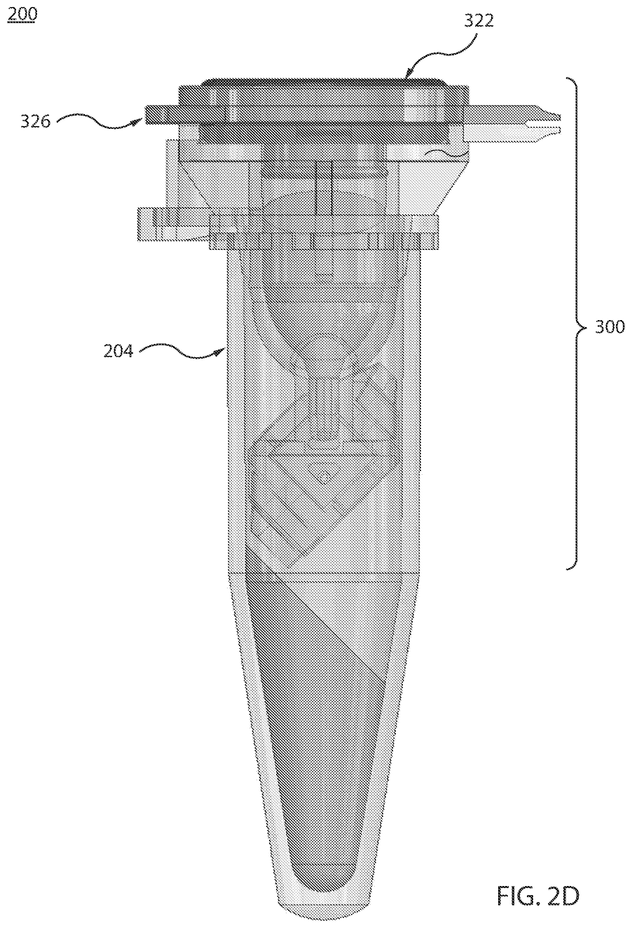

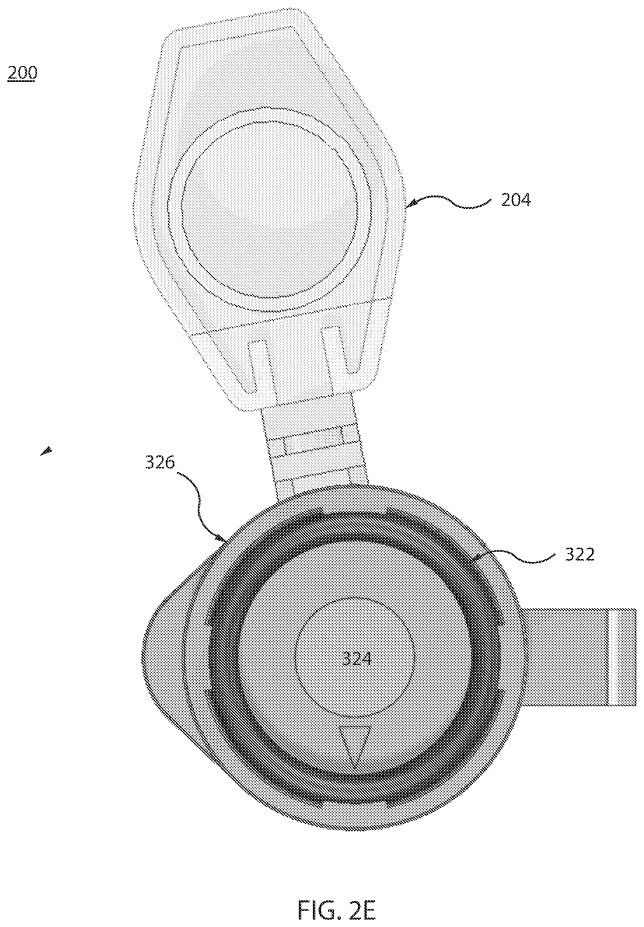

Below, exemplary embodiments of improved cartridges for use in systems for intracellular payload delivery, such as cartridge 300 for use in system 100, are described. FIGS. 2A-2E illustrate various views of device 200 including cartridge 300 and container 204, for use in system 100 for delivering a payload to a cell, in accordance with some embodiments.

Cartridge 300 may be configured to house one or more constriction-containing elements, such as a constricting filter (containing one or more constricting pores) or a constricting microfluidic chip (containing one or more constricting microfluidic channels). (Constricting filters in accordance with some embodiments are disclosed in application number WO/2017/041050A1, which is hereby incorporated by reference in its entirety.) It should be noted that, in some embodiments, a constricting microfluidic channel or a constricting pore may simply be referred to as a “constriction” or a “cell-deforming constriction.” A constriction-containing element may be any component, device, part, or the like having a channel, passage, or other opening (e.g., a constriction) having a smaller diameter than a cell of a cell suspension passing through the element, such that forcing the cell through the opening under pressure causes a perturbation in the membrane of the cell as the cell is constricted by the opening. In some embodiments, device 200 and/or cartridge 300 may include integrated constricting filters or microfluidic channels configured to constrict cells, while in some embodiments cartridge 200 may be configured to house distinct elements (e.g., chips or filters) that themselves include constricting pores or constricting microfluidic channels. In either case, device 200 and cartridge 300 may define part of the flow path of a system for delivering a payload to a cell, such as system 100, such that a cell suspension may flow toward and into cartridge 300, and such that the cell suspension may then flow through and out of cartridge 300 and toward and into container 204 (and/or into any other suitable downstream flow path components).

FIG. 2A shows a perspective view of device 200. FIG. 2B shows an exploded perspective view of device 200. FIG. 2C shows a first side view of device 200. FIG. 2D shows a second side view of device 200, rotated 90 degrees from the first side view shown in FIG. 2C. FIG. 2E shows a top view of device 200. In each of the views of FIGS. 2A-2E, device 200 is shown with a chip and gasket assembly inserted in receiving portion 306 of cartridge 300.

In each of the views in FIGS. 2A-2C, portions of cartridge 300 (e.g., cartridge body 302) and container 202 are shown as partially transparent. In some embodiments, some or all portions of cartridge 300 and/or container 202 may be transparent, translucent, or opaque.

In the embodiments shown in FIGS. 2A-2E, container 204 takes the form of a tube, such as a test tube, culture tube, sample tube, centrifuge tube, or the like. In some embodiments, container 204 may be an Eppendorf tube. In this way, container 204 may be an off the shelf component with the additional configurations to house the chip.

In some embodiments, container 204 may comprise one or more components made of metal, plastic, polymers, and/or glass. In some embodiments container 204 may comprise one or more components made of polycarbonate, polypropylene, and/or polymethyl methacrylate.

In some embodiments, the internal cavity of container 204 may have a volume of greater than or equal to 1 mL, 1.5 mL, 2 mL, 2.5 mL, 3 mL, or 3.5 mL. In some embodiments, the internal cavity of container 204 may have a volume of less than or equal to 1 mL, 1.5 mL, 2 mL, 2.5 mL, 3 mL, or 3.5 mL.

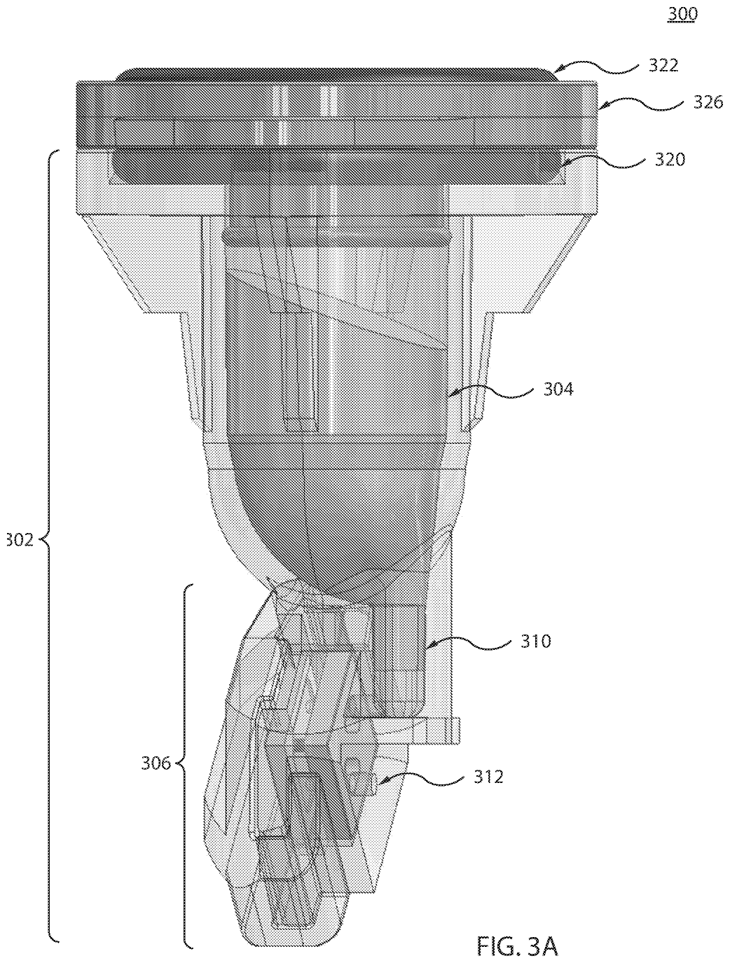

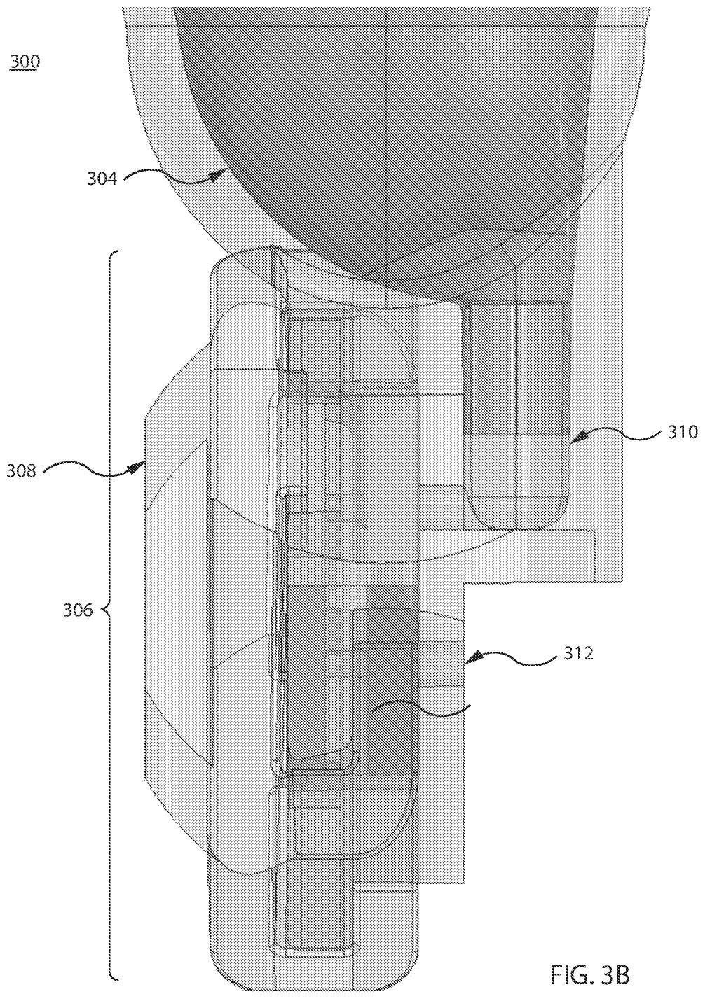

As further shown in FIGS. 2A-2E, cartridge 300 (shown and described in further detail with reference to FIGS. 3A-3B) may be configured to mate with the opening 205 of container 204, for example by taking the form of a cap for container 204. In some embodiments, cartridge 300 may be configured to mate with opening 205 of container 204 by a press connection, snap connection, twist-lock connection, or any other suitable connection. In some embodiments, cartridge 300 may be configured to be held against opening 205 of container 204 by one or more tube-handling devices, such as a robotic arm, during processing of cell suspension fluid through cartridge 300.

As described above, unlike cartridge systems that may be configured to be attached to an input reservoir by flexible tubing or by other fluidic components, the cartridges disclosed herein may include an input reservoir that is physically integrated with the cartridge itself. The input reservoir may be a reservoir configured to house cell suspension prior to its flow through the constriction-containing element. The cartridges disclosed herein may be provided in the form of a cap device configured to connect to and cover an opening of a container-such as a test tube, culture tube, sample tube, centrifuge tube, or the like. By using an integrated input reservoir, this eases integration with existing lab devices, such as readily available pressure sources. Additionally, the timing and potential volume reduction for using embodiments described herein may be particularly useful in a research use setting, where volumes are small, and consumables may be used quickly via numerous small volume experiments, for example. The container (e.g., tube) to which the cartridge may be attached as a cap may serve as an output reservoir for housing the cell suspension following processing through the constriction-containing element. In some embodiments, when the cartridge is in the attached position with the container (e.g., tube), a portion of the cartridge may be disposed inside an interior cavity of the container, for example by protruding downward into the interior of a test tube. This configuration eases the retrieval of a tube container, and may provide additional ease of use in a research setting.

Thus, when the cartridge is attached to an open end of a container such as a laboratory tube, the device formed by the combination of the cartridge and the container may include an input reservoir, a receiving portion configured to receive and hold the constriction-containing element, and an output reservoir provided in the form of the container such as a laboratory tube. All three components may be in fluid communication with one another to allow flow of the cell suspension from the input reservoir to the receiving portion (and into and out of a constriction-containing element housed therein) and then from the receiving portion to the container (e.g., tube). Following flow of the cell suspension into the container (e.g., tube), the cartridge may be detached from the opening of the container and the container (housing the processed cell suspension) may be used for further processing of the cell suspension, may be capped, and/or may be stored in any suitable manner. When the container is provided in the form of a standard laboratory tube, the tube may be easily used for downstream processing by one or more additional standard-compatible devices, caps, and/or storage devices.

As shown in FIGS. 2A and 2C-2E, cartridge 300 may be configured such that, when it is assembled with container 204 by being placed in an assembled position, cartridge body 302 extends downward into the interior cavity of container 204. In some embodiments, a width a portion of cartridge body 302 may be less than an interior diameter of the interior cavity of container 204, such that the portion of cartridge body 302 may extend downward into the interior cavity of container 204. In some embodiments, cartridge body 302 may include a rim portion that is wider than the portion that extends into the interior cavity of container 204, such that the rim portion may sit outside (e.g., sit on top of) opening 205 of container 204 while part of cartridge body 302 extends into the interior cavity of container 204. In some embodiments, when in the assembled position with container 204, cartridge 300 may extend into the interior cavity of container 204 by greater than or equal to 30%, 40%, 50%, 60%, or 70% of a total height dimension (in the direction in which cartridge 300 extends into the interior cavity) of the interior cavity of container 204. In some embodiments, when in the assembled position with container 204, cartridge 300 may extend into the interior cavity of container 204 by less than or equal to 30%, 40%, 50%, 60%, or 70% of a total height dimension (in the direction in which cartridge 300 extends into the interior cavity) of the interior cavity of container 204. In some embodiments, when in the assembled position with container 204, cartridge 300 may extend into the interior cavity of container 204 by greater than or equal 0.5 cm, 1 cm, 1.5 cm, 2 cm, or 2.5 cm. In some embodiments, when in the assembled position with container 204, cartridge 300 may extend into the interior cavity of container 204 by less than or equal 0.5 cm, 1 cm, 1.5 cm, 2 cm, or 2.5 cm.

In some embodiments, when in the assembled position with container 204, greater than or equal to 50%, 60%, 70%, 80%, 90%, 95%, or 99% by height of cartridge 300 may extend into the interior cavity of 204. In some embodiments, when in the assembled position with container 204, less than or equal to 50%, 60%, 70%, 80%, 90%, 95%, or 99% by height of cartridge 300 may extend into the interior cavity of 204.

As shown particularly in FIG. 2B, in some embodiments, cartridge 300 may include cartridge body 302 that includes input reservoir 304 and receiving portion 306. Input reservoir 304 may be configured to receive and hold a cell suspension before the cell suspension flows from input reservoir 304 to receiving portion 306.

In some embodiments, input reservoir may have a tapered shape that narrows at the bottom such that fluid may flow out of the bottom of input reservoir 304 into one or more channels under force of gravity and/or pressure (e.g., liquid pressure, gas pressure, and/or mechanical pressure. In some embodiments, input reservoir 306 may have an interior volume of less than or equal to 1 mL, 1.5 mL, 2 mL, 2.5 mL, or 3 mL. In some embodiments, input reservoir 306 may have an interior volume of greater than or equal to 1 mL, 1.5 mL, 2 mL, 2.5 mL, or 3 mL.

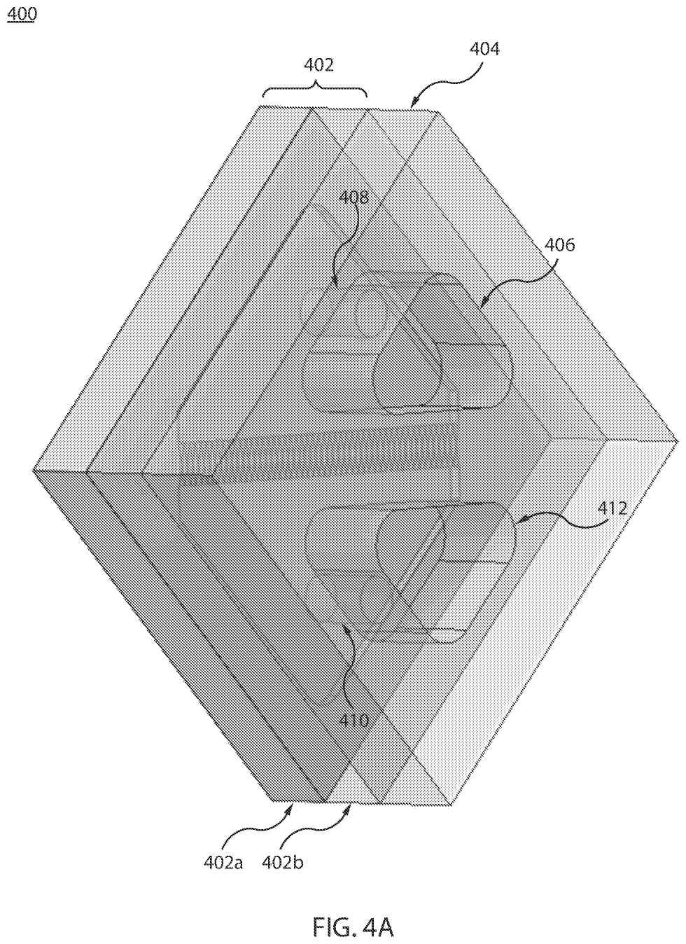

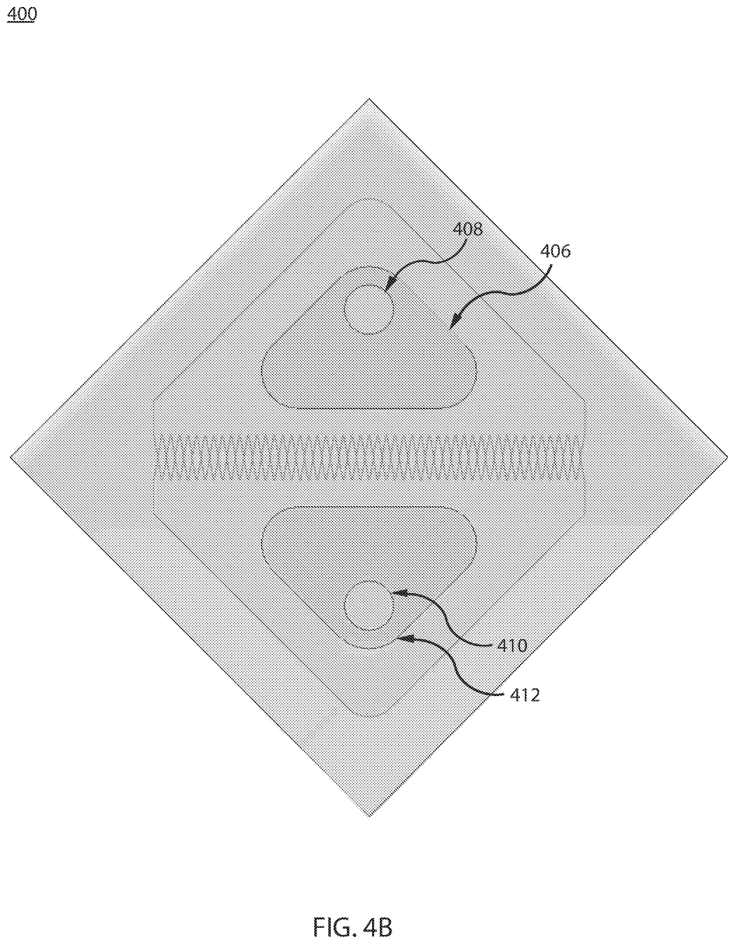

Receiving portion 306 may be configured to receive and hold a constriction-containing element, such as chip and gasket assembly 400 (shown and described in further detail with reference to FIGS. 4A-4B), and to allow flow of the cell suspension from input reservoir 304 into the receiving portion and through one or more channels in the receiving portion into the constriction-containing element. After the cell suspension has flowed through the constriction-containing element, the suspension may flow back into one or more channels of the receiving portion. The one or more channels of the receiving portion may be open to the exterior of cartridge 300, such that the cell suspension may flow out of cartridge 300 and into the interior cavity of container 204.

Cartridge 300 may comprise receiving portion cover 308, which may be configured to cover receiving portion 306 and to hold the constriction-containing element in place in receiving portion 306.

Receiving portion cover 308 may be an element configured to be placed alongside one or more constriction-containing elements, to press the one or more constriction-containing elements toward receiving portion 306, and/or to otherwise hold the one or more constriction-containing elements in place. In some embodiments, receiving portion cover 308 may be configured to apply inward force to one or more constriction-containing elements to press them toward receiving portion 306 by way of one or more springs or other compressible or deformable components, such as one or more rubber o-rings or gaskets. In some embodiments, receiving portion cover 308 may be configured to press flush against a surface of one or more of constriction-containing elements. In some embodiments, receiving portion cover 308 may serve to ensure that one or more constriction-containing elements do not delaminate a layer under the pressure of fluid being forced through them; by pressing receiving portion cover 308 against one face of a constriction-containing element under force, the constriction-containing element may be prevented from delaminating.

Receiving portion cover 308 may be attachable and removable from receiving portion 306, for example by a sliding connection, a threaded connection, a hinged connection, a tab-and-slot connection, a locking mechanism, by one or more screws, by one or more cams, or in any other suitable manner such that receiving portion cover 308 may be removed, for example, to replace gasket 404 and/or constriction-containing element 402.

In some embodiments, a sliding connection, such as the connection shown in FIGS. 2A-2E, may allow receiving portion cover 308 to slide laterally along receiving portion 306 in the (in the direction of the two-sided arrow shown in FIG. 2B), such that a lip of receiving portion cover 308 may slide onto a corresponding lip, protrusion, groove, or tooth of receiving portion 306. In some embodiments, a removable cover configured to slide onto and off of a body of a receiving portion may be configured to fully encircle a receiving portion, in some embodiments thereby avoiding the need for interlocking grooves or teeth or the like.

A sliding connection such as this may be removed with minimal lateral force (e.g., force in the direction of sliding), but may provide great strength in the direction perpendicular to the sliding direction and extending away from the receiving portion against which the cover is placed. Thus, while the cover having a sliding connection may be easily removed by hand, it may nonetheless offer superior durability under pressure to other connection mechanisms that may be used to hand-assemble cartridges, such attaching a cover by threaded components. In some embodiments, in addition to or alternately to one or more removable covers, a cartridge may be configured to securely house one or more constriction-containing elements without use of removable covers.

In some embodiments, receiving portion cover 308 may be configured to be slidably removable from receiving portion 306 such that it cannot be slidably removed from receiving portion 306 when cartridge 300 is in an assembled position with container 204. As shown in FIGS. 2A-2E, when cartridge 300 is assembled with container 204 such that cartridge body 302 extends downward into the interior cavity of container 204, the walls of container 204 may block receiving portion cover 308 from being able to slide off of either side of receiving portion 306. This arrangement may help to ensure that receiving portion cover 308 cannot become accidentally dislodged from receiving portion during operation.

Cartridge 300 may be configured such that cell suspension fluid may enter (e.g., may flow into) input reservoir 304 via an opening, such as a top opening 305 as shown in FIG. 2B. In some embodiments, input reservoir 304 may have an open top end such that cell suspension may be flowed into input reservoir 304 from the top.

In some embodiments, cartridge 300 may include a lid 326 that may cover an opening of input reservoir 304. Lid 326 may be configured to mate with the opening of input reservoir 304 by a press connection, snap connection, twist-lock connection, or any other suitable connection. Lid 326 may be a foldable lid comprising an upper portion 327 and a lower portion 329, wherein upper portion 327 may fold up into an open position to allow access to input reservoir 304 and then fold down into a closed position to close the cell suspension inside input reservoir 304. In some embodiments, lid 326 may be placed into the closed position before pressure is applied to the cell suspension inside input reservoir 304. In some embodiments, lid 326 may include filter 324, which may be configured to prevent liquid (e.g., cell suspension) from flowing back out of the cartridge toward a cell suspension source and/or a pressure source. Air pressure may be supplied above filter 324, e.g., from the pressure source.

As shown particularly in FIGS. 2B and 2C, cartridge 300 may include two or more o-rings including o-ring 320 and o-ring 322. O-ring 320 may be disposed between lid 326 and cartridge body 302, forming a seal therebetween. O-ring 322 may be disposed on an opposite side of lid 326 from cartridge 302, and may form a seal between lid 326 with another component of system 100, such as cell suspension source 108 and/or pressure source 110. In some embodiments, o-ring 322 may be pressed against another component of system 100 (e.g., by a robotic arm) in order to form a seal and allow positive pressure to be applied to the cell suspension inside input reservoir 304.

FIGS. 3A-3B illustrate views of a cartridge 300 for use in system 100 for delivering a payload to a cell, in accordance with some embodiments.

FIG. 3A shows a side view of cartridge 300. FIG. 3B shows a close-up side view of the lower portion of device 300, focusing on receiving portion 306 and receiving portion cover 308. In both FIGS. 3A and 3B, cartridge 300 is shown with a chip and gasket assembly inserted therein.

In both FIGS. 3A and 3B, portions of cartridge 300 (e.g., cartridge body 302) are shown as partially transparent. In some embodiments, some or all portions of cartridge 300 may be transparent, translucent, or opaque.

FIGS. 3A and 3B provide views of the interior channels formed within cartridge body 302 that facilitate flow of cell suspension through cartridge 300.

As shown, input channel 310 fluidly connects input reservoir 304 to receiving portion 306. In the arrangement shown, input channel 310 has an initial vertical portion and then a second horizontal portion, beginning at an outlet from input reservoir 304 and terminating at a portion of receiving portion 306 that is configured to hold a gasket for a constriction-containing element (discussed in further detail with respect to FIGS. 4A-4B). In some embodiments, all or part of input channel 310 may have a diameter of less than or equal to 0.05 mm, 0.1 mm, 0.5 mm, 1 mm, 1.5 mm, 2 mm. or 2.5 mm. In some embodiments, all or part of input channel 310 may have a diameter of greater than or equal to 0.05 mm, 0.1 mm, 0.5 mm, 1 mm, 1.5 mm, 2 mm. or 2.5 mm.

As shown, output channel 312 begins at a portion of receiving portion 306 that is configured to hold a gasket for a constriction-containing element, and terminates at an opening to the exterior of cartridge 300. Thus, cell suspension that has passed through the constriction-containing element may flow through output channel 312 and flow out of cartridge 300, for example dripping downward under force of gravity to be collected in a container (e.g., container 204) into which cartridge 300 is inserted. In some embodiments, all or part of output channel 312 may have a diameter of less than or equal to 0.05 mm, 0.1 mm, 0.5 mm, 1 mm, 1.5 mm, 2 mm. or 2.5 mm. In some embodiments, all or part of output channel 312 may have a diameter of greater than or equal to 0.05 mm, 0.1 mm, 0.5 mm, 1 mm, 1.5 mm, 2 mm. or 2.5 mm.

In some embodiments, cartridge 300 may be less than or equal to 0.5 mm, 0.6 mm, 0.75 mm, 1 mm, 5 mm, 1 cm, 5 cm, or 10 cm in height (e.g., in the vertical direction as shown in FIG. 2A). In some embodiments, cartridge 300 may be greater than or equal to 0.5 mm, 0.6 mm, 0.75 mm, 1 mm, 5 mm, 1 cm, 5 cm, or 10 cm in height.

In some embodiments, cartridge 300 may be less than or equal to in width (e.g., in the direction running horizontally in FIG. 2A) as measured at a portion of cartridge 300 that extends into the interior cavity of container 204 when in the assembled position with respect to container 204. In some embodiments, cartridge 300 may be less than or equal to in width as measured at a portion of cartridge 300 that extends into the interior cavity of container 204 when in the assembled position with respect to container 204.

FIGS. 4A-4B illustrate views of a chip and gasket assembly 400 for use with a cartridge for use in system 100 for delivering a payload to a call, in accordance with some embodiments. Chip and gasket assembly 400 may be the same chip-and-gasket assembly that is shown in FIGS. 2A-2D, 3A, and 3B. While FIGS. 4A-4B show an exemplary microfluidic chip comprising a plurality of constrictions configured to induce perturbations in cell membranes, it will be appreciated in light of the disclosure herein that a microfluidic filter configured to induce perturbations in cell membranes could additionally or alternatively be used.

FIG. 4A shows a perspective view of chip and gasket assembly 400. FIG. 4B shows a plan view of chip and gasket assembly 400 with gasket 404 in the foreground. In both FIGS. 3A and 3B, portions of cartridge 300 (e.g., cartridge body 302) are shown as partially transparent. In some embodiments, some or all portions of cartridge 300 may be transparent, translucent, or opaque.

As shown particularly in FIG. 4A, chip and gasket assembly 400 may comprise chip 402 and gasket 404, which may be disposed adjacent to one another in a layered arrangement. Chip 402 may itself comprise a constriction layer 402a and a cover layer 402b.

Constriction layer 402a may be a layer in which one or more cell-deforming constrictions are formed, for example by being etched or machined into the layer. Constriction layer 402a may, in some embodiments, be formed of silicon.

Cover layer 402b may be disposed against constriction layer 402a in order to cover one or more cavities and/or constrictions formed in constriction layer 402a. Cover layer 402b may thus form a “lid” on top of cover layer 402a and seal in the volumetric regions through which the cell suspension flows in constriction layer 402a. As shown in FIGS. 4A and 4B, cover layer 402a may comprise chip inlet channel 408 and chip outlet channel 410, each of which may be formed as through holes in cover layer 402a to allow cell suspension to flow through the channels. Cell suspension may flow into chip 402 and through chip inlet channel 408 into constriction layer 402a, and may flow out of constriction layer 402a and into chip outlet channel 410 and out of chip 402. Cover layer 402b may, in some embodiments, be formed of glass. Cover layer 402b may be bonded to constriction layer 402a by any suitable means, such as by adhesive.

In some embodiments, one or both of chip inlet channel 408 and chip outlet channel 410 may have a diameter of less than or equal to 0.2 mm, 0.4 mm, 0.6 mm, 0.8 mm, 1 mm, or 1.2 mm. In some embodiments, one or both of chip inlet channel 408 and chip outlet channel 410 may have a diameter of greater than or equal to 0.2 mm, 0.4 mm, 0.6 mm, 0.8 mm, 1 mm, or 1.2 mm.

Gasket 404 may be configured to be disposed against cover layer 402a. Gasket 404 may provide channels that fluidly connect chip 402 to the channels of receiving portion 306, forming a seal against receiving portion 306 and allowing fluid to flow from receiving portion 306 to chip 402 and to flow from chip 402 back to receiving portion 306.

As shown in FIGS. 4A and 4B, gasket 404 may comprise gasket inlet channel 406 and gasket outlet channel 412, each of which may be formed as through holes in gasket 404 to allow cell suspension to flow through the channels. Cell suspension may flow through gasket inlet channel 406 into chip 402, and may flow out of chip 402 into and through gasket outlet channel 412.

In some embodiments, one or both of gasket inlet channel 406 and gasket outlet channel 412 may have a cross-sectional dimension (e.g., fluid diameter) of from 0.1 mm to 2.0 mm.

Gasket 404 may, in some embodiments, be formed of one or more silicones, rubbers, and/or thermoplastic elastomers.

Thus, cell suspension may flow from input reservoir 304 to input channel 310, from input channel 310 to gasket inlet channel 406, from gasket inlet channel 406 to chip inlet channel 408, from chip inlet channel 408 to constriction layer 402a, from constriction layer 402a to chip outlet channel 410, from chip outlet channel 410 to gasket outlet channel 412, from gasket outlet channel 412 to output channel 312, and from output channel 312 out of cartridge 300 and into the interior cavity of container 204. Buffer fluid or other fluid may also flow along a same or similar flow path.

In some embodiments, in addition to or alternatively to using a gasket such as gasket 414, other sealing options may be used to create a seal for a fluid connection between a cartridge and a constriction-containing element; for example, o-rings, over-molding, chemical bonding, and/or mechanical interlocks may be used.

In some embodiments, chip 402 may have a length and/or width of less than or equal to 50 mm, 25 mm, 10 mm, 5 mm, 4 mm, 3 mm, 2 mm, or 1 mm. In some embodiments, chip 402 may have a length and/or width of greater than or equal to 50 mm, 25 mm, 10 mm, 5 mm, 4 mm, 3 mm, 2 mm, or 1 mm. In some embodiments, chip 402 may have a footprint of 4.1 mm by 4.1 mm

In some embodiments, chip 402 may have a thickness of less than or equal to 0.1 mm, 0.5 mm, 1 mm, 2 mm, 3 mm, or 5 mm. In some embodiments, chip 402 may have a thickness of less than or equal to 0.1 mm, 0.5 mm, 1 mm, 2 mm, 3 mm, or 5 mm.

In some embodiments, constriction layer 402a may have a thickness of less than or equal to 0.2 mm, 0.4 mm, 0.6 mm, 0.8 mm, 1 mm, 1.5 mm, or 2 mm. In some embodiments, constriction layer 402a may have a thickness of less than or equal to 0.2 mm, 0.4 mm, 0.6 mm, 0.8 mm, 1 mm, 1.5 mm, or 2 mm.

In some embodiments, cover layer 402b may have a thickness of less than or equal to 0.2 mm, 0.4 mm, 0.6 mm, 0.8 mm, 1 mm, 1.5 mm, or 2 mm. In some embodiments, cover layer 402b may have a thickness of less than or equal to 0.2 mm, 0.4 mm, 0.6 mm, 0.8 mm, 1 mm, 1.5 mm, or 2 mm.

In some embodiments, a constriction (e.g., a constricting channel or constricting pore) of a constriction-containing element (e.g., chip 402) may be less than or equal to 0.25 μm, 0.5 μm, 1 μm, 5 μm, 10 μm, 20 μm, or 50 μm in width. In some embodiments, a constriction (e.g., a constricting channel or constricting pore) of a constriction-containing element (e.g., chip 402) may be greater than or equal to 0.25 μm, 0.5 μm, 1 μm, 5 μm, 10 μm, 20 μm, or 50 μm in width.

In some embodiments, a constriction (e.g., a constricting channel or constricting pore) of a constriction-containing element (e.g., chip 402) may be less than or equal to 1 μm, 5 μm, 10 μm, 20 μm, 30 μm, 40 μm, 50 μm, or 100 μm in length. In some embodiments, a constriction (e.g., a constricting channel or constricting pore) of a constriction-containing element (e.g., chip 402) may be greater than or equal to 1 μm, 5 μm, 10 μm, 20 μm, 30 μm, 40 μm, 50 μm, or 100 μm in length.

In some embodiments, a constriction (e.g., a constricting channel or constricting pore) of a constriction-containing element (e.g., chip 402) may be less than or equal to 10 μm, 15 μm, 20 μm, 50 μm, 80 μm, 100 μm, or 200 μm in depth. In some embodiments, a constriction (e.g., a constricting channel or constricting pore) of a constriction-containing element (e.g., chip 402) may be greater than or equal to 10 μm, 15 μm, 20 μm, 50 μm, 80 μm, 100 μm, or 200 μm in depth.

In some embodiments, gasket 404 may have a length and/or width of less than or equal to 50 mm, 25 mm, 10 mm, 5 mm, 4 mm, 3 mm, 2 mm, or 1 mm. In some embodiments, gasket 404 may have a length and/or width of greater than or equal to 50 mm, 25 mm, 10 mm, 5 mm, 4 mm, 3 mm, 2 mm, or 1 mm. In some embodiments, gasket 404 may have a footprint of 4.1 mm by 4.1 mm.

In some embodiments, gasket 404 may have a thickness of less than or equal to 0.25 mm, 0.5 mm, 0.75 mm, 1 mm, 1.25 mm, 1.5 mm, 1.75 mm, 2 mm, or 2.25 mm. In some embodiments, gasket 404 may have a thickness of less than or equal to 0.25 mm, 0.5 mm, 0.75 mm, 1 mm, 1.25 mm, 1.5 mm, 1.75 mm, 2 mm, or 2.25 mm.

In some embodiments, a plurality of constriction-containing elements in cartridge 300 may be arranged in series, such that a flow path through cartridge 300 may be a single linear path. In some embodiments, a plurality of constriction-containing elements in cartridge 300 may be arranged in parallel, such that a flow path in cartridge 300 diverges into a plurality of parallel segments as fluid travels through cartridge 300, and may then re-converge before flowing out of cartridge 300. In some embodiments, three or more constriction-containing elements may be arranged in cartridge 300 such that one or more of the elements are in series with another of the constriction-containing elements and one or more of the elements are in parallel with another of the constriction-containing elements.

In some embodiments, cartridge 300 may be configured to be able to receive a blank placeholder element in place of a functional constriction-containing element, wherein the blank placeholder element may not contain any channels or pores, or may otherwise be configured to disallow flow through the portion of cartridge 300 housing the placeholder element. By using a blank placeholder element, cartridge 300 may cause flow of fluid through a smaller number of constriction-containing elements at a time, or through only one constriction-containing element at a time, such that the system need not be used at the maximum capacity of constriction-containing elements at all times.

In some embodiments, device 200 may be configured to force fluid through cartridge 300 at pressures of less than or equal to 1, 5, 10, 25, 50, 75, 100, 125, 150, or 200 PSI. In some embodiments, device 200 may be configured to force fluid through cartridge 300 at pressures of greater than or equal to 1, 5, 10, 25, 50, 75, 100, 125, 150, or 200 PSI. In some embodiments, device 200 may be configured to be used with constriction-containing elements (e.g., constriction-containing elements) that may each individually (e.g., on a “per chip” basis) provide a throughput of less than or equal to 50, 100, 150, 200, 250, 300, or 400 mL of red-blood-cell suspension per minute. In some embodiments, device 200 may be configured to be used with constriction-containing elements (e.g., constriction-containing elements) that may each individually (e.g., on a “per chip” basis) provide a throughput of greater than or equal to 50, 100, 150, 200, 250, 300, or 400 mL of red-blood-cell suspension per minute.

In some embodiments, device 200 may be configured to be used with constriction-containing elements (e.g., constriction-containing elements) that may each individually (e.g., on a “per chip” basis) provide a throughput of less than or equal to 25, 50, 75, 100, 125, 150, or 200 mL of peripheral-blood-mononuclear-cell suspension per minute. In some embodiments, device 200 may be configured to be used with constriction-containing elements (e.g., constriction-containing elements) that may each individually (e.g., on a “per chip” basis) provide a throughput of greater than or equal to 25, 50, 75, 100, 125, 150, or 200 mL of peripheral-blood-mononuclear-cell cell suspension per minute.

In some embodiments, device 200 may have an overall fluid throughput (e.g., including all constriction-containing elements housed in in cartridge 300) of less than or equal to 0.01 L/min, 0.05 L/min, 0.1 L/min, 0.25 L/min, 0.5 L/min, or 1 L/min. In some embodiments, device 200 may have an overall fluid throughput (e.g., including all constriction-containing elements housed in in cartridge 300) of greater than or equal to 0.01 L/min, 0.05 L/min, 0.1 L/min, 0.25 L/min, 0.5 L/min, or 1 L/min.

1. A device for facilitating delivery of a payload to cells of a cell suspension, the device comprising:
a container comprising an interior cavity and an opening; and
a cartridge configured to connect to the opening of the container when in an assembled position with respect to the container, the cartridge comprising:
an input reservoir configured to receive a cell suspension; and
a receiving portion configured to receive a constriction-containing element, wherein the constriction-containing element comprises a constriction configured to perturb membranes of cells of the cell suspension to facilitate delivery of a payload to the cells, wherein the receiving portion is fluidly connected to the input reservoir to allow flow of the cell suspension from the input reservoir to the receiving portion, and wherein the receiving portion is directly fluidly connected to a cartridge outlet port to allow flow of the cell suspension from the receiving portion into the interior cavity of the container.

2. The device of claim 1, wherein, a) when the cartridge is in the assembled position with respect to the container, a portion of the receiving portion of the cartridge is disposed inside the interior cavity of the container, and/or b) when the cartridge is in the assembled position with respect to the container, a portion of the input reservoir of the cartridge is disposed inside the interior cavity of the container.

3. (canceled)

4. The device of claim 1, further comprising a cover configured to releasably attach to the receiving portion of the cartridge to hold the constriction-containing element in place when the constriction-containing element is received by the receiving portion.

5.-7. (canceled)

8. The device of claim 1, wherein the cartridge comprises an input reservoir cover that is movable between an open position and a closed position to selectively expose and enclose an interior of the input reservoir.

9. The device of claim 8, wherein the cartridge comprises a) a first o-ring disposed against a surface of the input reservoir cover that faces away from the interior of the input reservoir when the input reservoir cover is in the closed position, and/or b) a second o-ring disposed between the input reservoir cover and a body portion of the cartridge.

10. (canceled)

11. The device of claim 8 wherein the input reservoir cover comprises a filter configured to prevent backflow of the cell suspension.

12.-13. (canceled)

14. A device for facilitating delivery of a payload to cells of a cell suspension, the device comprising:
a container comprising an interior cavity and an opening;
a cartridge configured to connect to the opening of the container when in an assembled position with respect to the container, the cartridge comprising:
an input reservoir configured to receive a cell suspension; and
a receiving portion; and
a constriction-containing element, wherein the constriction-containing element comprises a constriction configured to perturb membranes of cells of the cell suspension to facilitate delivery of a payload to the cells;
wherein the receiving portion of the cartridge is fluidly connected to the input reservoir and to the constriction-containing element, such that the cell suspension is allowed to flow from the input reservoir through the receiving portion and into the constriction-containing element, and such that the cell suspension is allowed to flow out of the constriction-containing element through the receiving portion and into the interior cavity of the container.

15. The device of claim 14, further comprising a gasket disposed between the receiving portion and the constriction-containing element.

16.-17. (canceled)

18. The device of claim 14, wherein, a) when the cartridge is in the assembled position with respect to the container, a portion of the receiving portion of the cartridge is disposed inside the interior cavity of the container, and/or b) when the cartridge is in the assembled position with respect to the container, a portion of the input reservoir of the cartridge is disposed inside the interior cavity of the container.

19. (canceled)

20. The device of claim 14, wherein the cartridge further comprises a cover configured to releasably attach to the receiving portion of the cartridge to hold the constriction-containing element in place when the constriction-containing element is received by the receiving portion.

21.-23. (canceled)

24. The device of claim 14, wherein the cartridge comprises an input reservoir cover that is movable between an open position and a closed position to selectively expose and enclose an interior of the input reservoir.

25. The device of claim 24, wherein the cartridge comprises a) a first o-ring disposed against a surface of the input reservoir cover that faces away from the interior of the input reservoir when the input reservoir cover is in the closed position, and/or b) a second o-ring disposed between the input reservoir cover and a body portion of the cartridge.

26. (canceled)

27. The device of claim 24 wherein the input reservoir cover comprises a filter configured to prevent backflow of the cell suspension.

28.-29. (canceled)

30. A cartridge for facilitating delivery of a payload to cells of a cell suspension, the cartridge comprising:
an input reservoir configured to receive a cell suspension;
a connection portion configured to connect to an opening to an interior cavity of a container; and
a receiving portion configured to receive a constriction-containing element, wherein the constriction-containing element comprises a constriction configured to perturb membranes of cells of the cell suspension to facilitate delivery of a payload to the cells, wherein the receiving portion is fluidly connected to the input reservoir to allow flow of the cell suspension from the input reservoir to the receiving portion, and wherein the receiving portion is fluidly connected to a cartridge outlet port to allow flow of the cell suspension out of the cartridge.

31. The cartridge of claim 30, wherein, a) when the cartridge is connected in an assembled position with respect to the container, a portion of the receiving portion of the cartridge is disposed inside the interior cavity of the container, and/or b) when the cartridge is in the assembled position with respect to the container, a portion of the input reservoir of the cartridge is disposed inside the interior cavity of the container.

32. (canceled)

33. The cartridge of claim 30, wherein the cartridge further comprises a cover configured to releasably attach to the receiving portion of the cartridge to hold the constriction-containing element in place when the constriction-containing element is received by the receiving portion.

34.-36. (canceled)

37. The cartridge of claim 30, wherein the cartridge comprises an input reservoir cover that is movable between an open position and a closed position to selectively expose and enclose an interior of the input reservoir.

38. The cartridge of claim 37, wherein the cartridge comprises a) a first o-ring disposed against a surface of the input reservoir cover that faces away from the interior of the input reservoir when the input reservoir cover is in the closed position, and/or b) a second o-ring disposed between the input reservoir cover and a body portion of the cartridge.

39. (canceled)

40. The cartridge of claim 37 wherein the input reservoir cover comprises a filter configured to prevent backflow of the cell suspension.

41. (canceled)

42. The cartridge of claim 30, wherein the cartridge is configured to allow flow of the cell suspension through the cartridge at a pressure of greater than 100 PSI and or less than 200 PSI.

43.-48. (canceled)
